(19) United States
(12) Patent Application Publication
(10) Pub. No.: US 2016/0287783 A1
(43) Pub. Date: Oct. 6, 2016

(54) IMPLANTABLE DRUG DELIVERY DEVICE WITH A CHECK VALVE

(71) Applicant: Beijing ZhaoShi Medical Equipment Co., Ltd., Beijing (CN)

(72) Inventors: Xiaoyun Zhao, Beijing (CN); Ying Bi, Beijing (CN)

(21) Appl. No.: 14/824,046

(22) Filed: Aug. 11, 2015

(30) Foreign Application Priority Data
Apr. 3, 2015 (CN) 201510158575.0

(51) Int. Cl. A61M 5/168; A61M 39/24

(52) U.S. Cl. CPC A61M 5/16813; A61M 39/24

(57) ABSTRACT

An implantable drug delivery device, comprising a check valve, a locking device, a drug delivery device body capable of being connected to a well-matched catheter through a locking device, wherein the drug delivery device body further comprising a pump frame, a well-matched punctured diaphragm disposed on the top of the pump frame, a pump cover well-matched with pump frame and punctured diaphragm disposed on the top of the punctured diaphragm, the pump cover, punctured diaphragm and pump frame sealingly connected to each other; and a check valve connected to the pump frame, wherein a pump cavity is formed by the pump cover, punctured diaphragm, pump frame and the check valve forming a pump cavity.

### CROSS-REFERENCE TO RELATED APPLICATIONS

The present application claims priority based on Chinese Application No. 201510158575.0 filed on Apr. 3, 2015, the disclosure of which is hereby incorporated by reference in its entirety.

### TECHNICAL FIELD

The present invention relates to the field of medical equipment, and particularly to an implantable drug delivery device comprising a check valve.

### BACKGROUND

In the present market, all kinds of catheters entered human body and need to stay in the body for more than one week. After the drugs being injected through catheter by medical personnel, the catheter needs to be sealed with heparin water. The outlet of the catheter is often blocked by thrombus. Sometimes, the improper operation of medical staff causes the heparin water to leak into the subcutaneous tissue and causes irritation in the patient. Such irritation can easily be mistaken for wound infection. This side effect can present complication in medical staff's work and make the device difficult to use. For the above problems in the related technologies, no effective solutions have been put forward currently.

### SUMMARY OF THE INVENTION

In view of the above problems in related technologies, the present invention relates to an implantable drug delivery device with check valve without sealing catheter to avoid drugs back flowing and clogging catheters effectively.

To achieve the above technical purpose, the technical scheme of the invention is that:

An implantable drug delivery device with a check valve without sealing catheter included a drug delivery device body, the drug delivery device body connected to a well-matched catheter through locking device, the drug delivery device body including pump frame, the top of the pump frame setting up a well-matched punctured diaphragm, the top of the punctured diaphragm setting up a pump cover well-matched with pump frame and punctured diaphragm, the pump cover, punctured diaphragm and pump frame seal connecting to each other, the pump frame connecting with a check valve, the pump cover, punctured diaphragm, pump frame and check valve forming pump cavity between them. Preferably, the locking device included joint coupling, the joint coupling setting up external thread well-matched with the pump frame, the catheter threaded joint coupling and connected with frustum cone type steel needle through locking inner sleeve, the frustum cone type steel needle containing inner hole.

Preferably, the check valve included one-way diversion switch set in the pump cavity and O Ring set on one-way diversion switch, the top of the one-way diversion switch containing through-hole, the through-hole and inner hole interlinked, the on one-way diversion switch, O Ring, frustum cone type steel needle and locking inner sleeve forming one-way flow of liquid.

The beneficial effects of the invention is that: using an implantable drug delivery device with a check valve without sealing catheter of this invention, not only the drug in artery pump can directly into the established parts to make targeted therapy effect, drug concentration increasing 10% to lesion and lethality of diseased cells can improving about four times of the therapeutic efficacy; but also it can effectively avoid drugs back flowing to clogged catheters, and then avoid the medical personnel using heparin water to seal catheter after injection of drugs for patients, effectively improving the practicability of the device.

### BRIEF DESCRIPTION OF THE DRAWINGS

Further features and advantages of the present device, as well as the structure and operation of various embodiments of the present device, will become apparent and more readily appreciated from the following description of the preferred embodiments, taken in conjunction with the accompanying drawings of which:

FIG. 1 is a breakdown structure diagram of an implantable drug delivery device with a check valve without sealing catheter according to the present invention; and

FIG. 2 is a diagrammatic cross-section of an implantable drug delivery device with a check valve without sealing catheter according to the present invention; and

FIG. 3 is a structure diagram of a frustum cone type steel needle according to the present invention; and

FIG. 4 is a structure diagram of one-way diversion switch according to the present invention.

In the diagram:
1, pump frame;
2, punctured diaphragm;
3, pump cover;
4, pump cavity;
5, locking inner sleeve;
6, joint coupling;
7, external thread;
8, catheter;
9, a frustum cone type steel needle;
10, inner hole;
11, one-way diversion switch;
12, O Ring;
13, through-hole.

### DETAILED DESCRIPTION

It will clearly and completely illustrate the technical proposal according to the appended drawings in this invention example in the following. Obviously, the implementation described below is just some cases of the present invention, and all the other cases obtained by technicians in this field according to the case in this invention are within the protection scope of the present invention.

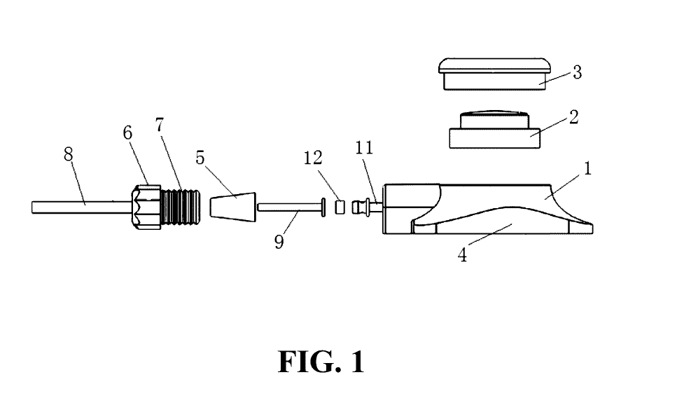

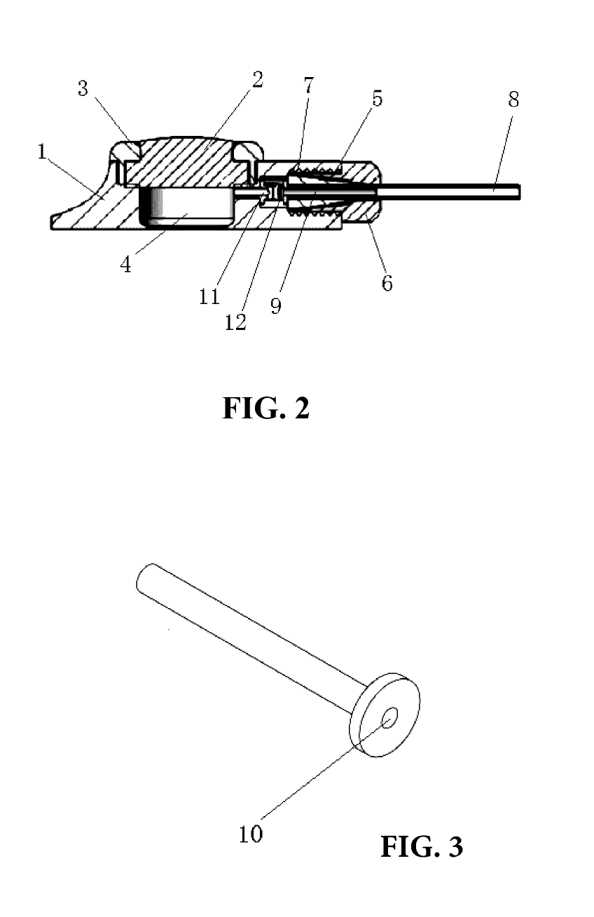

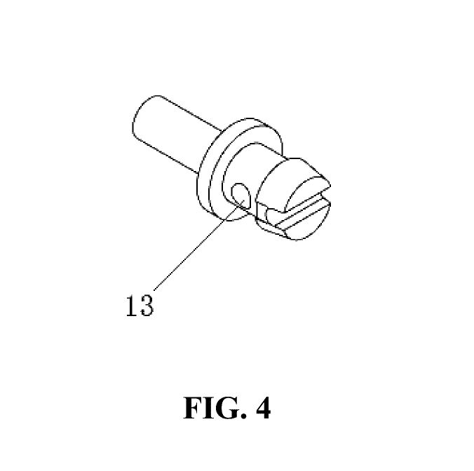

Referring to FIGS. 1 and 2, an implantable drug delivery device with a check valve without sealing catheter according to the implementation in this invention included a drug delivery device body, the drug delivery device body connected to a well-matched catheter 8 through a locking device, the drug delivery device body including pump frame 1, a well-matched punctured diaphragm 2 is disposed on top of the pump frame 1, a pump cover 3 is disposed on top of the punctured diaphragm 2, the pump cover 3 is well-matched with pump frame 1 and punctured diaphragm 2 , the pump cover 3, punctured diaphragm 2 and pump frame 1 are sealingly connected to each other, the pump frame 1 is connected to a check valve, the pump cover 3, punctured diaphragm 2, pump frame 1 and locking device together forms pump cavity 4 between them.

Furthermore, in one particular embodiment, the locking device included joint coupling 6, the joint coupling 6 setting up external thread 7 well-matched with the pump frame 1, the catheter 8 threaded Joint coupling 6 and connected with frustum cone type steel needle 9 through locking inner sleeve 5, the frustum cone type steel needle 9 containing inner hole 10.

Furthermore, in one particular embodiment, the check valve included one-way diversion switch 11 set in the pump cavity 4 and O Ring 12 set on one-way diversion switch 11, the top of the one-way diversion switch 11 containing through-hole 13, the through-hole 13 and inner hole 10 interlinked, the on one-way diversion switch 11, O Ring 12, frustum cone type steel needle 9 and locking inner sleeve 5 forming one-way flow of liquid.

In order to easy to understand the above technical scheme of the present invention, specific means using the above technical scheme of the invention in detail will be explained in the following.

When used in concrete, the soup injected into pump cavity 4, with the amount of the soup increasing, the pressure increasing in the pump cavity 4 until reach a certain degree, the soup transited through T-type channel of one-way diversion switch 11, with opening O Ring 12, and then the soup is injected into external cavity of pump frame. Since through-hole 13 setting up on the top of one-way diversion switch 11 is connected to the inner hole of frustum cone type steel needle 9, and around of external cavity of pump frame sealed with locking inner sleeve 5. The soup enters frustum cone type steel needle 9 via through-hole 13, and then enters the human body via catheter 8, which connected to frustum cone type steel needle 9.

When the drug is stopped, O Ring 12 rebounded and blocking the T-type channel of one-way diversion switch 11, return channel of the soup is closed to ensure that the soup does not return.

And the interventional catheter needs a ribbon to enter, and the tip of the catheter has an opening, and an opening can't prevent the back flow of blood from the artery. So minimally invasive approach is unable to carry out the targeted therapy of the artery without the technology of check valve without SEALING CATHETER implantable. We have developed the technology of the one-way valve-free sealing device, which fills the gaps in the domestic technology to obtain the method of minimally invasive surgery being implanted in artery.

All this lead to that using an implantable drug delivery device with a check valve without sealing catheter of this invention, not only the drug in artery pump can directly into the established parts to make targeted therapy effect, drug concentration increasing 10% to lesion and lethality of diseased cells can improving about four times of the therapeutic efficacy; but also it can effectively avoid drugs back flowing to clogged catheters, and then avoid the medical personnel using heparin water to seal catheter after injection of drugs for patients, effectively improving the practicability of the device.

The above is just the preferred embodiment of the invention, and not to limit the invention. Any changes made, equivalent replacement, improvement shall be included in the scope of protection of the invention within the spirits and principles of the present invention.

What is claimed is:

1. An implantable drug delivery device, comprising:
a check valve,
a locking device,
a drug delivery device body capable of being connected to a well-matched catheter through a locking device, wherein the drug delivery device body further comprising a pump frame, a well-matched punctured diaphragm disposed on the top of the pump frame, a pump cover well-matched with pump frame and punctured diaphragm disposed on the top of the punctured diaphragm, the pump cover, punctured diaphragm and pump frame sealingly connected to each other; and a check valve connected to the pump frame, wherein a pump cavity is formed by the pump cover, punctured diaphragm, pump frame and the check valve forming a pump cavity.

2. An implantable drug delivery device as set forth in claim 1, wherein the locking device comprising a joint coupling, a frustum cone type steel needle, and a locking inner sleeve, wherein the joint coupling further comprising external threads well-matched with the pump frame , wherein the frustum cone type steel needle further comprising an inner hole and is capable of being connected with the catheter through the join coupling and the locking inner sleeve.

3. An implantable drug delivery device as set forth in claim 1, wherein the check valve comprising an one-way diversion switch set inside the pump cavity, and an O Ring, wherein the one-way diversion switch further comprising a through hole, wherein the one-way diversion switch is connected with inner hole; wherein the O Ring is set on the one-way diversion switch, wherein the one-way diversion switch, the O Ring, the frustum cone type steel needle and the locking inner sleeve together causes an one-way flow of liquid.
